(19) United States
(12) Patent Application Publication
(10) Pub. No.: US 2026/0097228 A1
(43) Pub. Date: Apr. 9, 2026

(54) A HIGH FREQUENCY MAGNETIC INDUCTION DEVICE FOR THE TREATMENT OF ORAL CANCER

(71) Applicant: MAGHEALS PRIVATE LIMITED, Bangalore, Karnataka (IN)

(72) Inventors: Shilpee JAIN, Bangalore, Karnataka (IN); Rajesh KATOCH, Bangalore, Karnataka (IN); Kaushik SUNEET, Bangalore, Karnataka (IN)

(21) Appl. No.: 19/120,813

(22) PCT Filed: Oct. 13, 2023
(86) PCT No.: PCT/IB2023/060354

§ 371 Date: Apr. 14, 2025

(30) Foreign Application Priority Data
Oct. 13, 2022 (IN) 202241022075

(51) Int. Cl. A61N 2/02; A61K 9/10; A61K 31/695; A61K 33/26; A61N 2/00

(52) U.S. Cl. CPC A61N 2/02; A61K 9/10; A61K 31/695; A61K 33/26; A61N 2/002; A61N 2/004

(57) ABSTRACT

The invention relates to a high frequency magnetic induction device (100). The device (100) comprises magnetic nanosuspension (101) and an AMF generator (103). The AMF generator (103) further comprises copper induction coil (104), work head (105), flexible connector (106), power supply (107), chiller (108), display and control unit (109), fiberoptic thermometer sensors (110a & 110b), auto-manual toggle switch (111), USB port (112a & 112b) and bed support (113). Tumor site enclosed by the magnetic nanosuspension (101) comprising iron oxide magnetic nanoparticles (102) generates heat when exposed to AMF generated by the AMF generator (103). The heat generated does not cause any harm to the healthy tissues. The AMF generator (103) has precise control over thermal doses generated. The high frequency induction device (100) is safe, effective, non-invasive with mild side-effects.

### PRIORITY CLAIM

The application claims priority from the provisional specification numbered 202241022075 filed with the Indian Patent Office, Chennai on 13 Apr. 2022 post-dated to 13 Oct. 2023 entitled “A high frequency magnetic induction device for the treatment of oral cancer”, the entirety of which is expressly incorporated herein by reference.

### PREAMBLE TO THE DESCRIPTION

The following specification particularly describes the invention and the manner in which it is to be performed:

### DESCRIPTION OF THE INVENTION

### Technical Field of the Invention

The present invention discloses a high frequency magnetic induction device for the treatment of oral cancer. More particularly, the high frequency magnetic induction device comprises a nanosuspension comprising iron oxide nanoparticles and an alternating magnetic field (AMF) generator. The high frequency magnetic induction device is non-invasive, safe with mild side effects for the reduction of tumor size.

### BACKGROUND OF THE INVENTION

Oral cancer relates to cancer that develops in the tissues of mouth. They belong to group of head and neck cancers. Oral cancer is also known as mouth cancer. The tumor of oral cancer develops in cheeks, tongue, palate, lips, or gums. Oral cancer is one of the most common types of cancer which is often undetected.

The symptoms of oral cancer are painful mouth ulcers, persistent lumps in the buccal cavity, numbness, lisp etc. The types of oral carcinoma are squamous cell carcinoma, adenocarcinoma, sarcoma, malignant melanoma, and lymphoma. The most common type of oral cancer is oral squamous cell carcinoma.

The major risk factors affecting oral cancer are smoking and drinking alcohol. They have a synergistic effect in causing oral cancer in most of the cases. The tobacco present in cigarettes is carcinogenic, which causes oral cancer. The passive smokers i.e., people who do not smoke but exposed to smoke in the surrounding environment are also at a risk of developing the cancer. The oral immunity is weakened due to smoking. Consumption of alcohol is a systemic and local risk factor for oral cancer. Chronic use of alcohol results in impaired immune system and increased susceptibility to oral cancer. The exposure to UV radiation and Human Papilloma Virus (HPV) are among other risk factors.

There are various treatment modalities available for treatment of oral cancer including surgery, radiotherapy, and chemotherapy. However, combination treatments are often provided to prevent the recurrence of cancer. The treatment regime depends on the location, size, stage of the primary tumor and the comorbidities. The incidence of oral malignancies has increased over the years. Due to the aggressive invasion and metastasis, the possibility of recurrence is high. Post-treatment the diagnosis and prognosis of tumor reduces drastically.

The oral cancer treatments mainly focus on primarily preserving function and appearance of the mouth. Most of the oral cancer detections are in later stages, which imposes difficulty in curing the cancer. Oral cancer causes considerable impairment in speech, chewing and swallowing. The potential non-invasive treatments for oral cancer are targeted therapy and immunotherapy that possess various risks.

The current treatment options for oral cancer comes with various unwanted off-target side effects. These side effects reduce the quality of life. However, thermal ablation is a potential and promising alternative cancer treatment.

Thermal ablation involves applying heat at the site of cancer, the applied heat causes cell damage and eventually necrosis of tumor cells. The selective heat delivery at the tumor site is a challenge. Magnetic nanoparticles (MNP) are used for targeted delivery of heat to the tumor cells. The MNP emit thermal energy when they are exposed to alternating magnetic field called as Magnetic Hyperthermia. The magnetic hyperthermia therapy has proven to be beneficial for unresectable tumors.

The Patent Application No. WO2021US42257 titled “Magnetic hyperthermia treatment systems and methods” discloses a magnetic hyperthermia treatment for treating a patient with tumor. The magnetic hyperthermia treating system comprises a conducting particle of 500 microns and oscillating magnetic field for heating the conducting particle. The method comprises placing a conducting particle within the tumor of the patient, wherein the conducting particle has a diameter with a value within a range from about 20 microns to about 1000 microns and heating the conducting particle with an oscillating magnetic field. The conducting particle is placed within the tumor by biopsy needle, a bone marrow syringe, and a standard syringe. The conducting particle of the system is attached with a filament with low conductivity. The conducting particle comprises gold and titanium materials. The system for treating tumor comprises a conducting particle placed within the tumor of the patient, a magnetic field generation device, and a computer system in operative association with the magnetic field generation device.

The Patent Application No. WO2018IB00218 titled “Magnetic field oscillating at several frequencies for improving efficacy and/or reducing toxicity of magnetic hyperthermia” discloses use of magnetic nanoparticles for magnetic hyperthermia. The magnetic hyperthermia therapeutic treatment, prophylactic treatment, diagnosis method, comprises magnetic nanoparticles that are administered to a body part of an individual and the body part is exposed to a magnetic field oscillating at a high frequency and at a medium and/or low frequency, wherein the high frequency is 1 MHz at the most, the medium frequency is lower than the high frequency, and the low frequency is lower than the high frequency and lower than the medium frequency when it is present. The high frequency range is between 1-1000 kHz which heats the magnetic nanoparticles. The heating step produces a temperature increase of more than 1° C. of the body part. The cooling step induces a temperature decrease of more than 1° C. of the body part. The magnetic field comprises cycles with heating and cooling steps. The parameters considered for the selection of magnetic nanoparticles are average or maximum magnetic field amplitude, magnetic field strength, magnetic field amplitude, magnetic field frequency, and spatial or temporal distribution of magnetic field lines. The magnetic nanoparticles are used for the prevention and treatment of cancer, tumor and an infection.

Oral cancer is one of the most common types of cancer and often goes undetected. The chances of recurrence of oral cancer despite taking treatment is high. The side-effects due to prevailing treatment options are several. The treatment of oral cancer using hyperthermia provides localized treatment and reduces the chances of recurrence. The state of art providing hyperthermia therapies have various shortcomings such as invasive delivery of heat energy, heat sink effect, and incomplete ablation. There is a need for a device to provide optimum heat delivery conditions along with maximum efficacy and minimal side effects.

### SUMMARY OF THE INVENTION

The present invention discloses a high frequency magnetic induction device which overcomes the drawbacks of the existing prior arts. The present invention comprises a nanosuspension comprising iron oxide magnetic nanoparticles and alternating magnetic field generator aiding in reduction of tumor size.

The present invention discloses a high frequency magnetic induction device comprising nanosuspension, iron oxide magnetic nanoparticles, alternating magnetic field (AMF) Generator, copper induction coil, work head, flexible connector, power supply, chiller, auto-manual toggle switch, display and control unit, fiberoptic thermometer sensors, USB port and bed support.

The iron oxide nanoparticles of the nanosuspension upon exposure to alternating magnetic field generates heat by the mechanism of hysteresis and magnetic relaxation loss. The generated heat subjected to a tumor aid in reduction of the tumor size. The generated heat results in increased internal temperature of the tumor area causing damage to the cancerous cells eventually leading to cell death.

The present invention discloses a high frequency magnetic induction device aiding in the reduction of tumor size. The heat generated by the iron oxide nanoparticles does not cause any harm to the surrounding healthy tissues. The AMF generator has precise control over thermal doses generated. The high frequency induction device is portable, safe, effective, biocompatible, non-invasive with minimal side-effects.

### BRIEF DESCRIPTION OF DRAWINGS

FIG. 1 illustrates the high frequency magnetic induction device.

FIG. 1a illustrates the high frequency magnetic induction device according to an embodiment of the invention.

FIG. 2 illustrates a flow chart for the induction of magnetic field in the magnetic nanosuspensions.

FIGS. 3a & 3b illustrate the X-ray diffraction spectra of the iron oxide nanoparticles of the nanosuspension upon application of alternating magnetic field.

FIG. 4 illustrates Fourier transform infrared (FTIR) spectrum of the coated and uncoated nanosuspension.

FIG. 5 illustrates the magnetic hysteresis (M-H) curves of the iron oxide magnetic nanoparticles.

FIG. 6 and FIG. 7 illustrate the heating efficiency of the nanosuspension comprising iron oxide magnetic nanoparticles.

FIG. 8 illustrates the power dependence and the position dependence of the heating efficiency of iron oxide magnetic nanoparticles.

FIG. 9 illustrates cellular viability of L929 and HepG2 cell lines upon incubation with the iron oxide magnetic nanoparticles.

FIG. 10 and FIG. 17 illustrate the distribution of nanosuspension in a tissue section.

FIG. 11 illustrates the ex vivo heating efficacy of the nanosuspension.

FIG. 12, FIG. 13 & FIG. 14 illustrates the biodistribution of nanosuspension.

FIG. 15 illustrates the histopathological imaging of kidney of the rat administered with nanosuspension.

FIG. 16 illustrates the transmission electron microscopy (TEM) images of the iron oxide magnetic nanoparticles.

### DETAILED DESCRIPTION OF THE INVENTION

In order to make the matter of the invention clear and concise, the following definitions are provided for specific terms used in the following description.

The term “Magnetic Nanoparticles” refers to a class of nanoparticles that can be altered using magnetic field.

The term “Hysteresis Loss” refers to the energy in magnetic materials, which is exposed to a magnetic field in the form of residual magnetization.

The term “Magnetic relaxation” refers to the Neel relaxation and Brownian motion of the magnetic nanoparticles when exposed to alternating magnetic fields.

The present invention discloses a high frequency magnetic induction device for the treatment of oral cancer. The high frequency magnetic induction device further comprises nanosuspension and an alternating magnetic field (AMF) generator. The nanosuspension comprises iron oxide magnetic nanoparticles. Iron oxide magnetic nanoparticles generate heat when exposed to AMF. The heat generated is utilized for the reduction of tumor size.

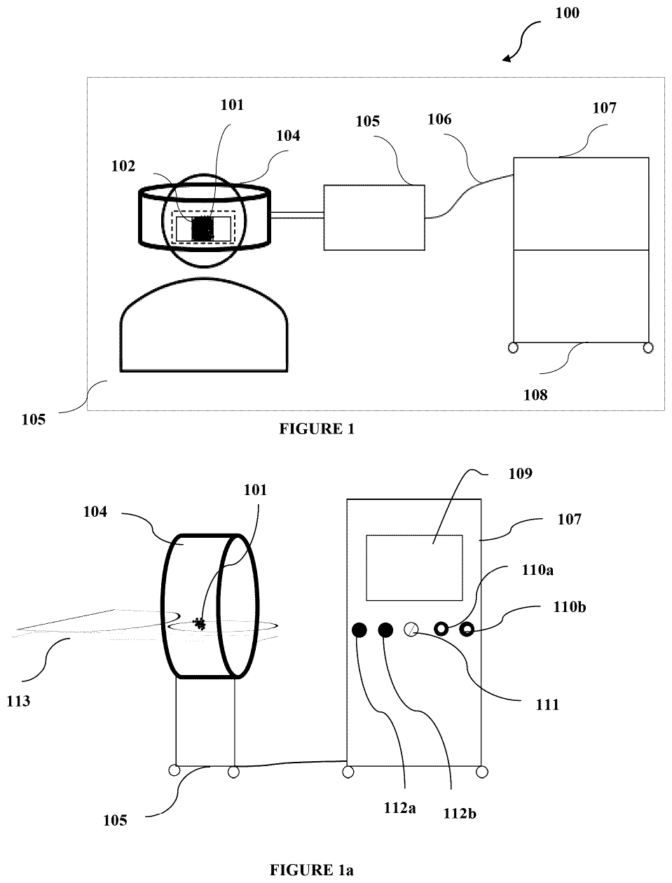

FIG. 1 illustrates the high frequency magnetic induction device (100). The high frequency magnetic induction device (100) comprises a nanosuspension (101) and an AMF generator (105). The nanosuspension (101) further comprises a iron oxide (Fe₃O₄) magnetic nanoparticles (102). The AMF generator (103) further comprises a copper induction coil (104), a working head (105), a flexible connector (106), a power supply (107) and a chiller (108).

FIG. 1a illustrates the high frequency magnetic induction device according to an embodiment of the invention. The device (100) comprises an AMF generator (103) and nanosuspension (101). The device (100) comprises a bed support (113) where the patient lies down horizontally. The tumorous area of the patient is enclosed by the nanosuspension (101) comprising iron oxide magnetic nanoparticles (102) surrounded by the copper induction coil (104). The copper induction coil (104) is of the diameter 300 mm. The copper induction coil (104) is connected to the working head (105) on one end of the device (100). The working head (105) is connected to the power generator (107) on the other end of the device (100). The power generator (107) comprises an auto-manual toggle switch (111) embedded to switch on or off the device (100) manually and automatically. The power generator (107) further comprises a display and control unit (109) embedded to display and regulate the input and output parameters. The power generator (107) further comprises fiberoptic thermometer sensors (110a & 110b) and USB ports (112a & 112b) embedded.

The device (100) generates a frequency of 50-100 kHz and a magnetic field intensity at a range of 0-500 G. The device (100) aids in the modification of the magnetic field intensity generated hence regulating the temperature range at the tumor site. The fiberoptic thermometer sensors (112a & 112b) aid in monitoring minute temperature changes crucial for the tumor.

The device (100) aids in the management of tumors by the mechanism of magnetic hyperthermia. Magnetic hyperthermia is induced by subjecting a magnetic material to an alternating magnetic field to generate heat. The action of heat aids in reduction of the tumor size. The magnetic materials of the size range greater than 20 nm generates heat by the mechanism of hysteresis loss.

The nanosuspension (101) comprises magnetic iron oxide magnetic nanoparticles (102) incorporated in them. The exposure of magnetic iron oxide magnetic nanoparticles (102) to the magnetic field generated by the AMF generator (103) results in generation of heat. The generation of heat is dependent on the size of magnetic material due to the mechanism of hysteresis and magnetic relaxation loss.

The iron oxide magnetic nanoparticles (102) exhibit hysteresis loss when placed in the magnetic field because of the alignment of magnetic dipole moments towards the external magnetic field. The reorientation of magnetic moment absorbs energy that dissipates in the form of heat. Brownian motion induces frictional heating due to the interaction between the nanoparticles and the surrounding medium. The generated heat is utilized for treating the tumour.

The nanosuspension (101) comprising iron oxide magnetic nanoparticles (102) is placed in the cancerous site and exposed to the alternating magnetic field in the copper coil (104) generated by the AMF generator (103). The generation of heat due to hysteresis and relaxation loss results in elevated localized temperature within a few seconds. The heat generated is dependent on magnetic field intensity, time, and concentration of magnetic nanoparticles. The increase in temperature around the tumor area kills the cancerous cells. The tumor does not dissipate heat to surrounding cells due to compact and unorganized vasculature. The ruptured vasculature prevents the supply of nutrients and blood to the tumor cells, resulting in irreversible cellular damage. At the cellular level, heat directly destroys the cancerous cells by damaging deoxy ribose nucleic acid (DNA) and interfering with DNA repair pathways. Moreover, the distribution of nanoparticles throughout the tumor generates uniform heat and destroys cancerous stem cells and hypoxic cells, which are unaffected by chemotherapy and radiation and are the major cause of recurrence.

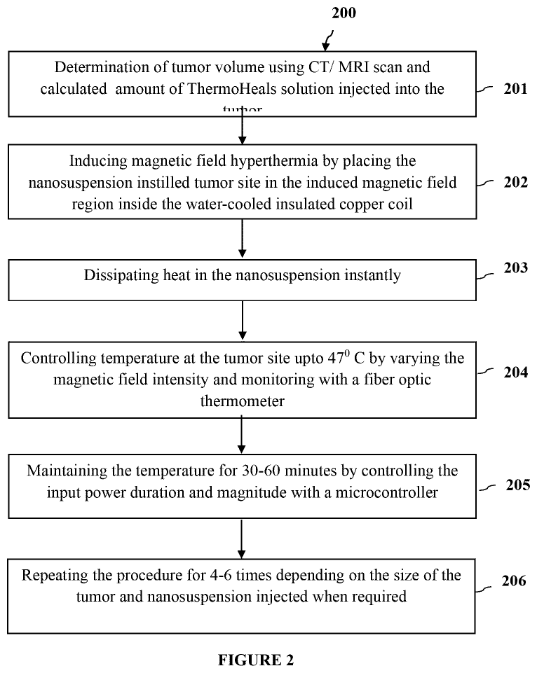

FIG. 2 illustrates a flow chart for the process of induction of magnetic field in the nanosuspension. The process of induction of magnetic hyperthermia (200) begins with a step of (201) where the nanosuspension (101) is injected into the tumor. The quantity of the nanosuspension (101) is obtained by the determination of tumor volume with computed tomography (CT) and magnetic resonance imagining (MRI) scans. At step (202), magnetic field hyperthermia is induced by placing the nanosuspension instilled tumor site in the induced magnetic field inside the water-cooled insulated copper coil (104). At step (203), heat is dissipated in the nanosuspension (101) instantly. At step (204), the temperature at the tumor site is controlled upto 47° C. by varying the magnetic field intensity and monitoring with a fiber optic thermometer (110a & 110b). At step (205), the temperature is maintained for 30-60 minutes by controlling the input power duration and magnitude with a microcontroller. At step (206), the procedure is repeated 4-6 times depending on the size of the tumor and the nanosuspension (101) is injected when required.

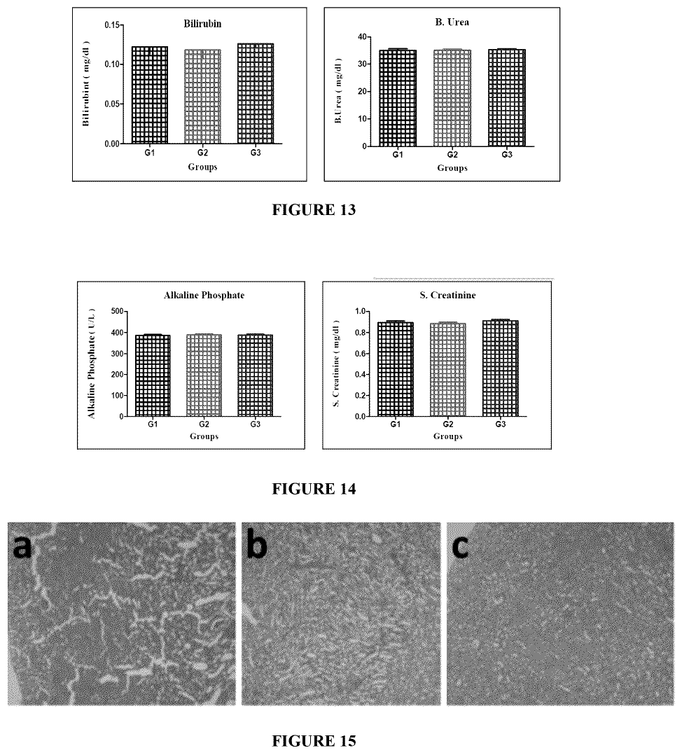

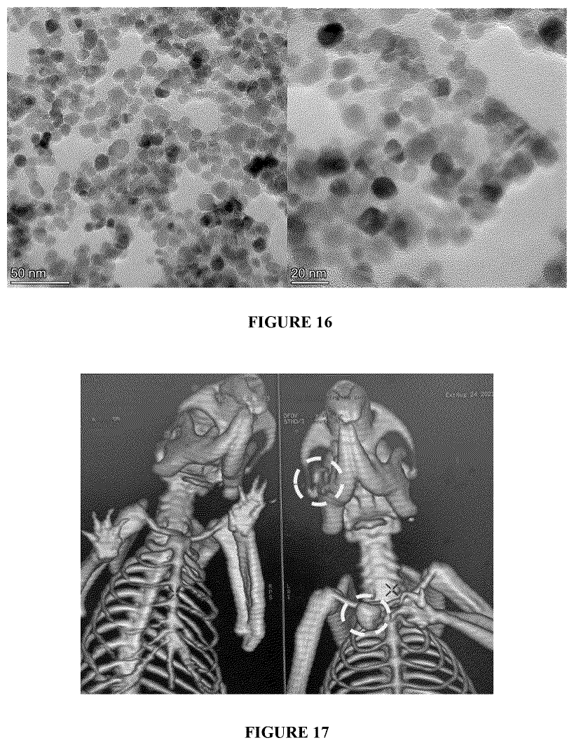

The present invention discloses a nanosuspension (101) comprising iron oxide magnetic nanoparticles (102). The nanosuspension (101) comprising iron oxide magnetic nanoparticles (102) enables uniform targeted heat delivery at the site of tumor. The iron oxide magnetic nanoparticles (102) are coated with alkoxysilane and suspended in distilled water. The average hydrodynamic size of the iron oxide magnetic nanoparticles (102) of the nanosuspension (101) is of the range 50 nm to 200 nm. FIG. 15 illustrates the transmission electron microscopy (TEM) images of the iron oxide nanoparticles. The TEM images aid in determining the magnetic core diameter of the iron oxide nanoparticles (102). The magnetic core diameter of the iron oxide nanoparticles (102) is at a range of 5 nm to 20 nm. The iron oxide nanoparticles (102) of the nanosuspension (101) exhibits polydispersity index at a range of 0.1 to 0.3. The zeta potential of the iron oxide nanoparticles (102) of the nanosuspension (101) is at a range of 30 mV to 55 mV. The iron oxide nanoparticles (102) of the nanosuspension are of absolute purity.

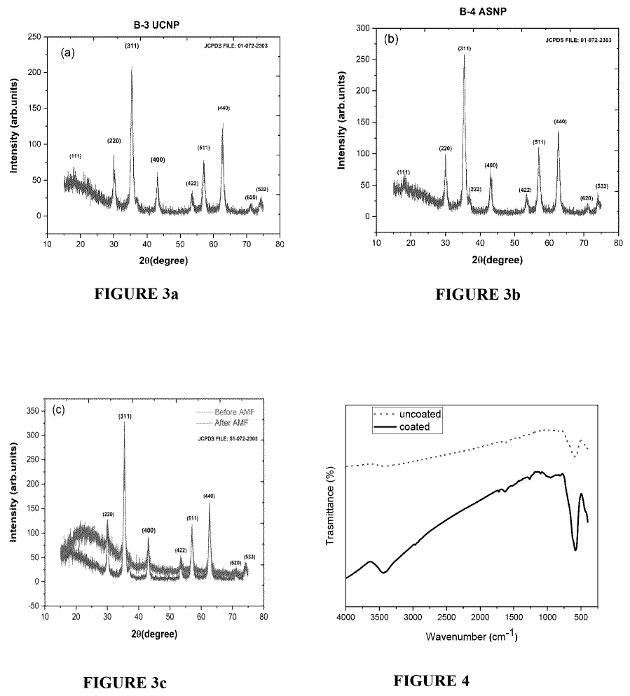

FIGS. 3a, 3b & 3c illustrate the X-ray diffraction spectra of the iron oxide nanoparticles of the nanosuspension upon application of alternating magnetic field. The phase purity and the absence of impurity is confirmed by the formation of secondary peaks.

FIG. 4 illustrates Fourier transform infrared (FTIR) spectrum of the coated and uncoated nanosuspension. The bands wavelength ranging from 1050 cm⁻¹ to 1110 cm⁻¹ indicates the Si—O and C—N bond respectively suggesting the presence of silane coating in coated particles and absent in uncoated particles.

The band at wavelength of 900 cm⁻¹ corresponds to bending vibration of the —NH₂ group and the band corresponding to Fe—O vibrations are observed around wavelength of 620 cm⁻¹.

FIG. 5 illustrates the magnetic hysteresis curves of the iron oxide nanoparticles. The hysteresis curve indicates the linear magnetization of the iron oxide nanoparticles (102). The coated iron oxide nanoparticles (102) prevent agglomeration and aids in alignment with the changing magnetic field.

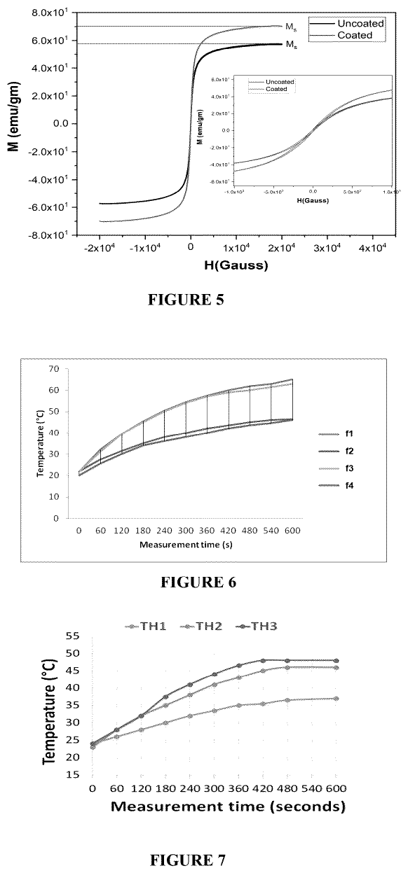

FIG. 6 and FIG. 7 illustrate the heating efficiency of the nanosuspension comprising iron oxide nanoparticles. The analysis of heating efficiency as a function of frequency of the alternating magnetic field radiation and concentration aids in determining the biosafety of the nanosuspension (101). FIG. 8 illustrates the power dependence and the position dependence of the heating efficiency of iron oxide nanoparticles. The increase in power increases the heating efficiency of the iron oxide nanoparticles (102). As magnetic hyperthermia is a non-contact process, the regulation of therapeutic temperature by maintaining constant frequency is crucial. The maintenance of constant power and frequency is useful in treatment planning such that it helps in positioning the tumor site at a zone of uniform magnetic fields and excluding the zone of higher magnetic field strength to provide effective therapeutic outcome.

Having generally described this invention, a further understanding can be obtained by reference to certain specific examples, which are provided herein for purposes of illustration only and are not intended to be limiting unless otherwise specified.

### Example 1: In Vitro & In Vivo Analysis Toxicity, Distribution, Heating Efficacy of the Nanosuspension

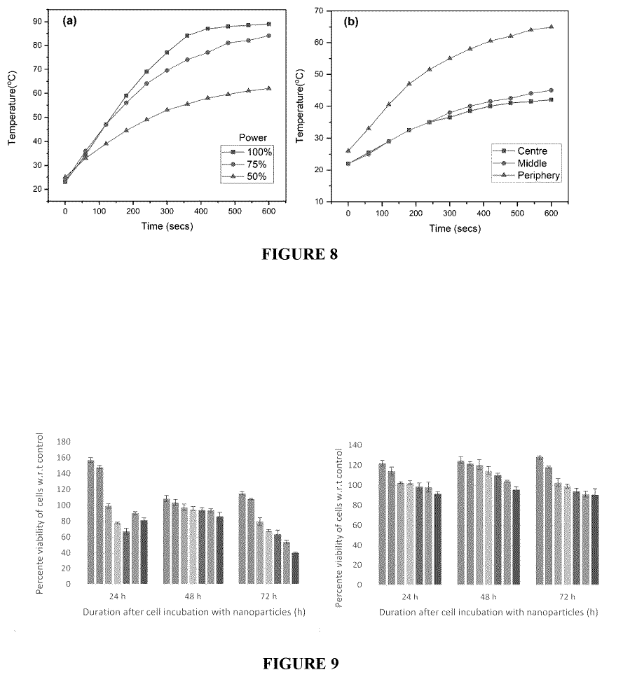

The nanosuspension (101) comprising iron oxide nanoparticles (102) was evaluated for toxicity by determining the cellular viability of L929 and HepG2 cell lines. The cell lines did not exhibit any toxicity after a duration of 3 days. FIG. 9 illustrates cellular viability of L929 and HepG2 cell lines upon incubation with the iron oxide nanoparticles. The absence of toxicity indicates the biocompatibility of nanosuspension.

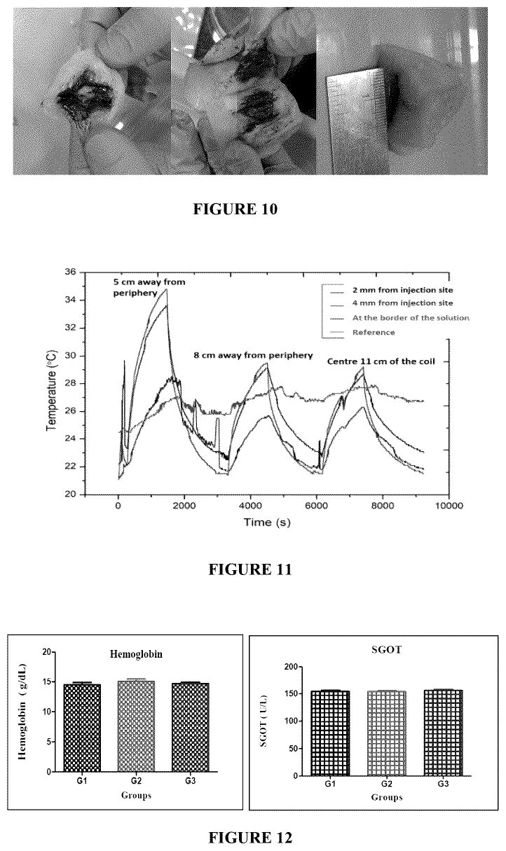

FIG. 10 and FIG. 17 illustrate the distribution of nanosuspension in a tissue section. The distribution of nanoparticles at the site of injection is dependent on the viscosity, the volume of the liquid, the flow rate of injection, and sites of injection of the nanosuspension (101). The nanosuspension (101) comprising iron oxide nanoparticles (102) was injected at a concentration of ˜50 μL of nanoparticles solution into the tumor induced in the head and neck area of hamsters using chemical carcinogens, and the distribution was confirmed by a computed tomography (CT) scan. The iron oxide magnetic nanoparticles (102) were observed at the site even after 24 h of the injection and cleared after six days of injection. Moreover, the animals were given two 30-minute hyperthermia sessions on alternate days. As the iron oxide magnetic nanoparticles (102) stayed at the site, the second session was given without further nanoparticle administration. The tumor of the size of around 10 mm was reduced after 2 sessions of the administration of the nanosuspension (101).

FIG. 12, FIG. 13 & FIG. 14 illustrates the biodistribution of nanosuspension. The biodistribution of nanosuspension was analyzed using Wistar rat animal model. The animal study comprised 3 groups of animals, including one control group and 2 test groups with different were tested. The nanosuspension was injected intravenously, and the distribution and accumulation of nanoparticles in different organs of all the animals were analyzed by using various blood parameters and histopathology after sacrificing the animals. FIG. 15 illustrates the histopathological imaging of kidney of the rat administered with nanosuspension. The histopathological and biodistribution study indicated there were no mortality, morbidity, abnormal behaviour, signs of biological reactivity and decrease in the body weights in any of the subject animals. The haematology, clinical chemistry, parameters were observed to be unaffected at the treated dose levels of test substance. There was no sign of accumulation of iron oxide magnetic nanoparticles (101) in any of the organs.

The present invention relates to a high frequency magnetic induction device (100) comprising nanosuspension (101) comprising iron oxide nanoparticles (102) and an AMF generator (105). The nanosuspension (101) is instilled into the tumour site and exposed to AMF generated by the AMF generator (105). The iron oxide magnetic nanoparticles (102) generate heat when exposed to AMF. The heat generated causes damage to the cancerous cells eventually killing the cancerous cells. The heat generated does not harm to surrounding tissues killing only the cancerous cells. The AMF generator (105) has precise control over thermal doses generated. The magnetic hyperthermia treatment is non-invasive, safe with mild side effects and suitable for human use. The device is safe, effective, biocompatible.

### REFERENCE NUMBERS

| Components | Reference Numbers |
|---|---|
| High frequency magnetic induction device | 100 |
| Nanosuspension | 101 |
| Iron oxide magnetic nanoparticles | 102 |
| AMF Generator | 103 |
| Copper Induction Coil | 104 |
| Work Head | 105 |
| Flexible Connector | 106 |
| RF Power Supply | 107 |
| Chiller | 108 |
| Display and control unit | 109 |
| Fiberoptic thermometer sensors | 110a & 110b |
| Auto- manual toggle Switch | 111 |
| USB port | 112a & 112b |
| Bed support | 113 |

We claim:

1. A high frequency magnetic induction device, the device comprising an AMF generator and nanosuspension:
i) the AMF generator (103) comprising:
a) a bed support (113) to allow the patient administered with a nanosuspension (101) comprising iron oxide magnetic nanoparticles (102) to lie down horizontally enclosed in a copper induction coil (104);
b) a copper induction coil (104) enclosing the tumor site connected to a working head (105) on one end to generate alternating magnetic field;
c) the working head (105) connected to the copper induction coil (105) on one end and a power generator (107) on the other end;
d) a display and control unit (109) embedded on the power generator (107) to display and regulate the input and output parameters;
e) the power generator (107) comprising an auto-manual switch (111) embedded to switch ON or OFF manually and automatically;
f) at least two fiberoptic thermometer sensors (110a & 110b) embedded on the power generator (107) to monitor and regulate the range of the temperature at the tumor site; and
g) at least two USB ports (112a & 112b) embedded on the power generator (107) for communication and interfacing with controlling software.
ii) the nanosuspension (101) comprising:
a) the iron oxide magnetic nanoparticles (102).

2. The device (100) as claimed in claim 1, wherein the nanosuspension (101) comprising iron oxide nanoparticles (102) is coated with alkoxysilane generated heat upon exposure to alternating magnetic field.

3. The device (100) as claimed in claim 1, wherein the average size of the iron oxide nanoparticles (102) of the nanosuspension (101) is of the range 50 nm to 200 nm.

4. The device (100) as claimed in claim 1, wherein the magnetic core diameter of the iron oxide nanoparticles (102) is at a range of 5 nm to 20 nm.

5. The device (100) as claimed in claim 1, wherein the iron oxide nanoparticles (102) of the nanosuspension (101) exhibits polydispersity index at a range of 0.1 to 0.3.

6. The device (100) as claimed in claim 1, wherein the zeta potential of the iron oxide nanoparticles (102) of the nanosuspension (101) is at a range of 30 mV to 55 mV.

7. The device (100) as claimed in claim 1, wherein a frequency of 50-100 kHz and a magnetic field intensity at a range of 0-500 G is generated to induce magnetic hyperthermia at the tumor site enclosed with the nanosuspension (101) comprising iron oxide nanoparticles (102).
